(19) United States
(12) Patent Application Publication
(10) Pub. No.: US 2011/0218388 A1
(43) Pub. Date: Sep. 8, 2011

(54) Ring Magnet for Obesity Management

(75) Inventors: Geoffrey R. Ball, Axams (AT); Dominik Hammerer, Innsbruck (AT)

(73) Assignee: VIBRANT MED-EL HEARING TECHNOLOGY GMBH, Innsbruck (AT)

(21) Appl. No.: 13/038,039

(22) Filed: Mar. 1, 2011

Related U.S. Application Data
(60) Provisional application No. 61/309,637, filed on Mar. 2, 2010.

(51) Int. Cl. A61F 2/04

(52) U.S. Cl. 600/30; 600/37

(57) ABSTRACT

A system and method for restricting an anatomical passageway includes a plurality of ring magnet pieces. Each of the plurality of ring magnet pieces has an inner and outer surface, and a pair of magnetic end sections between the outer surface and the inner surface, each end section having a characteristic magnetic field arrangement. An insert includes at least one of a magnet and a magnetic material. The ring magnet pieces are capable of forming, due to their magnetic field arrangements, an enclosed ring around an anatomical passageway. The insert is magnetically held in place by the ring magnet pieces when positioned in the anatomical passageway.

### CROSS-REFERENCE TO RELATED APPLICATIONS

This application claims priority to U.S. Provisional Patent Application No. 61/309,637 filed Mar. 2, 2010, which is hereby incorporated by reference herein in its entirety.

### TECHNICAL FIELD

The present invention relates to a system and method for restricting an anatomical passageway, and more particularly to a system and method of obesity management that includes a ring magnet in combination with an insert.

### BACKGROUND ART

Gastric bands are one option to treat obesity. Unlike gastric bypass and stomach stapling, gastric bands do not require cutting into the stomach and do not use any staple lines. The gastric band is wrapped around the upper portion of the patient's stomach, creating a stoma, or small pouch at the top of the stomach. The band slows the passage of food from the pouch to the lower part of the stomach, such that the pouch quickly fills with food. The filling of the upper portion of the stomach with food provides the sensation that the entire stomach is full. Consequently, the patient eats smaller portions, resulting in weight loss over time.

The gastric band often requires anchoring around the stomach by suturing together the ends of the gastric band, providing an initial sizing of the stoma. Furthermore, the inner diameter of the gastric band defining the passageway of food through the stomach and the size of the stoma often needs adjustment to achieve optimal restriction. The gastric band can not be so loose to where hunger is not controlled, nor overly tight to prevent sufficient consumption of food. One common way of adjustment, in addition to a latch that sets the diameter of the gastric band, is to inflate the gastric band via a port that is sutured or stapled under the skin to avoid infection. When saline is introduced into the band it expands, further restricting the patient's stomach. Less complex methods of attaching and adjusting the gastric band would be advantageous.

### SUMMARY OF THE INVENTION

Various embodiments of the invention are directed to a system and method for restricting an anatomical passageway. The system and method may be used, for example, to treat obesity, incontinence or disorders of the larynx.

In accordance with an embodiment of the invention, a system for restricting an anatomical passageway includes a plurality of ring magnet pieces. Each of the plurality of ring magnet pieces has an inner and outer surface, and a pair of magnetic end sections between the outer surface and the inner surface, each end section having a characteristic magnetic field arrangement. An insert includes at least one of a magnet and a magnetic material. The ring magnet pieces are capable of forming, due to their magnetic field arrangements, a ring around an anatomical passageway. The insert is magnetically held in place by the ring magnet pieces when positioned in the anatomical passageway.

In accordance with related embodiments of the invention, each magnetic field arrangement may include a plurality of magnetic field directions. At least one of the ring magnet pieces may include magnets arranged in an anti-parallel configuration. The insert may include an insert passageway having an inlet and an outlet. The system may include a plurality of the inserts, each insert having a passageway diameter that varies from the other inserts. The insert may include a tether to assist in extraction and/or installation.

In accordance with related embodiments of the invention, the insert may include a plurality of magnets. The magnets may be in an anti-parallel configuration. The plurality of magnets in the insert may be held by a material that dissolves within the anatomical passageway, whereupon the magnets of the insert are capable of changing position so as to increase the outer diameter of the insert. For example, the outer diameter of the insert may increase upon the material dissolving and being magnetically attracted to the ring magnet pieces. The plurality of magnets may be arranged in an iris pattern capable of moving from a first position in which the insert has a reduced outer diameter to a second position in which the insert has an increased outer diameter. The insert may be encapsulated in a rubber.

In accordance with another embodiment of the invention, a method for restricting an anatomical passageway includes placing a plurality of ring magnet pieces around an anatomical passageway such that due to their magnetic field arrangements they are held together to form an enclosed ring around the anatomical passageway. A first insert is inserted in the anatomical passageway, the first insert including at least one of a magnet and a magnetic material such that the first insert is magnetically held in place by, the ring magnet pieces.

In accordance with related embodiments of the invention, each ring magnet piece may have an inner and outer surface, and a pair of magnetic end sections between the outer surface and the inner surface, each end piece having a characteristic magnetic field arrangement. Each magnetic field arrangement may include a plurality of magnetic field directions. The insert may include an insert passageway having an inlet and an outlet.

In accordance with still further embodiments of the invention, the method further includes removing the first insert magnetically held in place by the ring magnet pieces, and inserting a second insert into the anatomical passageway such that it is magnetically held in place by the ring magnet pieces. The second insert may have an insert passageway that has a diameter different from the first insert.

In accordance with yet further embodiments of the invention, a tether associated with the first insert may be used to assist extraction and/or installation. The first insert may includes a plurality of magnets. The magnets of the first insert may be arranged in an anti-parallel configuration. The plurality of magnets in the first insert may be held by a material that dissolves within the anatomical passageway, wherein the outer diameter of the first insert increases upon the material dissolving. The first insert may include a material that dissolves within the passageway, whereupon the outer diameter of the first insert is capable of increasing.

### BRIEF DESCRIPTION OF THE DRAWINGS

The foregoing features of the invention will be more readily understood by reference to the following detailed description, taken with reference to the accompanying drawings, in which:

FIG. 1 shows a system for treating obesity, in accordance with an embodiment of the invention;

FIG. 2 shows a top view of a ring magnet, in accordance with an embodiment of the invention;

FIG. 3 shows a cross-sectional view of a ring magnet, in accordance with an embodiment of the invention;

FIG. 4 shows two ring magnet pieces and being drawn together by magnetic attraction to form a ring magnet, in accordance with an embodiment of the invention;

FIG. 5 shows a cross-sectional view of an insert that works in combination with ring magnet, in accordance with an embodiment of the invention;

FIG. 6 shows a ring magnet and insert being positioned relative to the stomach, in accordance with an embodiment of the invention;

FIG. 7 shows a ring magnet enclosed around the stomach, in accordance with an embodiment of the invention;

FIG. 8 shows varying diameters of inserts associated with an obesity management system, in accordance with an embodiment of the invention;

FIGS. 9(a) and 9(b) show ring magnet and insert configurations that may be advantageously used in situations where an MRI is required, in accordance with an embodiment of the invention;

FIG. 10 shows an insert shaped to aid installation, in accordance with an embodiment of the invention;

FIG. 11 shows an insert with an extraction tether, in accordance with an embodiment of the invention;

FIG. 12 shows an insert that includes a rubber encapsulation, in accordance with an embodiment of the invention;

FIG. 13 shows an insert that includes a dissolvable material, in accordance with an embodiment of the invention; and

FIG. 14 shows an insert that includes iris shaped elements that include magnets, in accordance with an embodiment of the invention.

### DETAILED DESCRIPTION OF SPECIFIC EMBODIMENTS

Illustrative embodiments are directed to a system and method for restricting an anatomical passageway. Generally, the system includes a ring magnet that is positioned around the anatomical passageway, and an insert that when inserted in the passageway is held in place by the ring magnet. The system and method may be used, for example, to treat obesity, incontinence or disorders of the larynx. Details are described below.

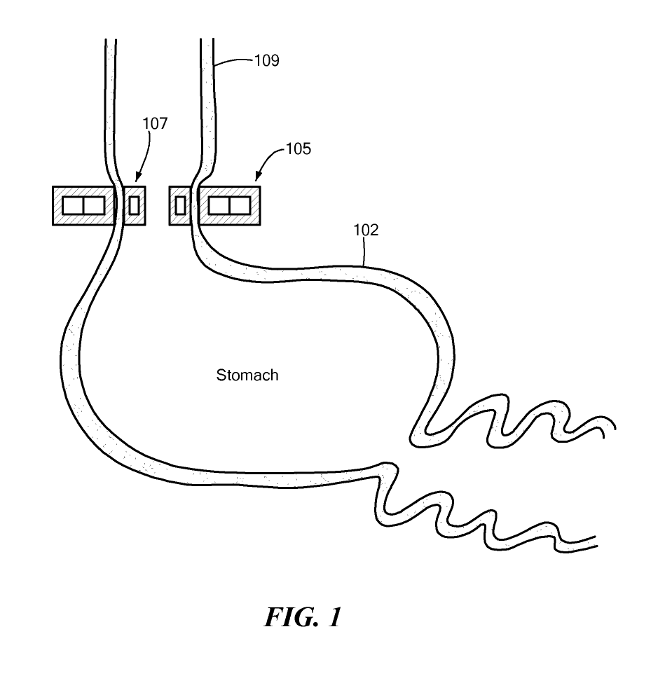

FIG. 1 shows a system for treating obesity, in accordance with an embodiment of the invention. The system includes a ring magnet 105 that is positioned around the stomach 102 of a patient. An insert 107 is inserted, for example, down through the esophageal tract 109. The insert includes a magnet(s) and/or a magnetic material, such as stainless steel, such that the insert 107 is magnetically held in place by the ring magnet 105 when positioned proximate the ring magnet 105.

The ring magnet 105, in combination with the insert 107, restricts the passageway of the stomach 102, allowing for limited passage of food, and in various embodiments, creating a stoma at the top of the stomach 102. Similar to a gastric band, the filling of the upper portion of the stomach 102 with food provides the sensation that the entire stomach 102 is full. Consequently, the patient eats smaller portions, resulting in weight loss over time.

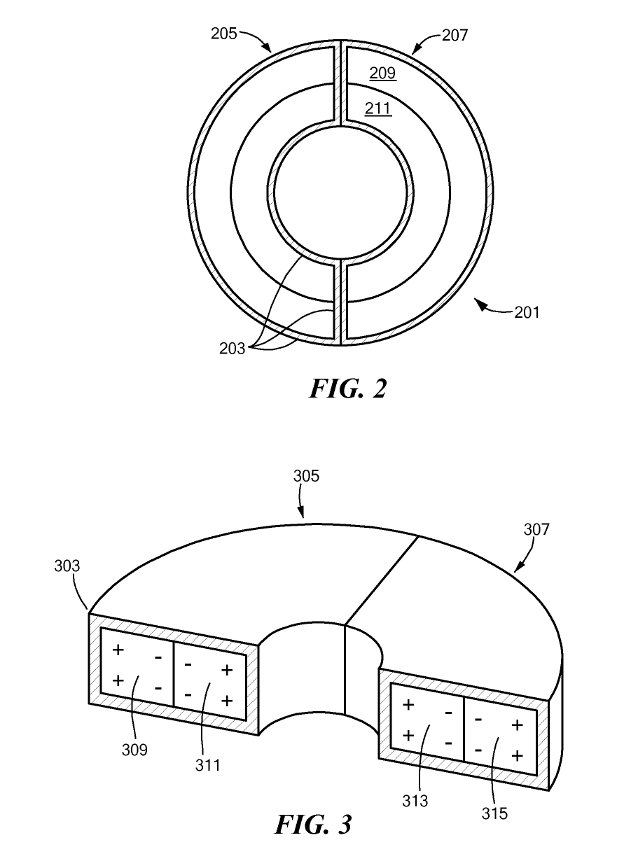

FIG. 2 shows a top view of a ring magnet 201 in more detail, in accordance with an embodiment of the invention. The ring magnet 201 is divided into a plurality of ring magnet pieces 205 and 207. Each ring magnet piece includes one or more magnets 209 and 211. The ring magnet pieces, when magnetically held together, form an opening. Each ring magnet piece 205 and 207 may include an enclosure 203, which may be, without limitation, a titanium enclosure or other biocompatible material.

FIG. 3 shows a cross-sectional view of a ring magnet, in accordance with an embodiment of the invention. The ring magnet includes two ring magnet pieces 305 and 307, each having a titanium enclosure 303. Ring magnet piece 305 includes magnets 309 and 311, while ring magnet piece 307 includes magnets 313 and 315.

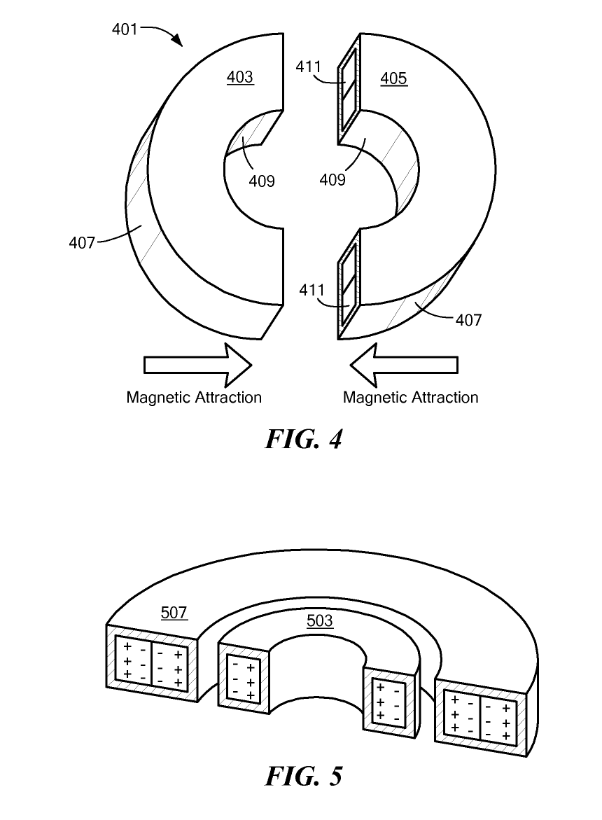

FIG. 4 shows two ring magnet pieces 403 and 405 being drawn together by magnetic attraction to form a ring magnet, in accordance with an embodiment of the invention. Each ring magnet piece 403 and 405 includes an outer surface 407 and an inner surface 409 and a pair of magnetic end sections 411 between the outer and inner surface 407 and 409, such that each end piece 411 has a characteristic magnetic field arrangement. The ring magnet pieces cooperate due to their magnetic field arrangements to form a ring for placing around an anatomical passageway.

FIG. 5 shows a cross-sectional view of an insert 503 that works in combination with ring magnet 507, in accordance with an embodiment of the invention. The insert 503 includes one or more magnets and/or magnetic material, such as stainless steel. Due to the magnet and/or magnetic material, the insert 503 is magnetically held in place by the ring magnet 507 when positioned within the opening formed by the ring magnet 507. The insert 503 typically includes, but is not limited to, a single component, which may include a biocompatible housing and the magnet(s) or magnetic material. The insert 503 may be advantageously shaped to form a passageway through which food or other substances may pass. The passageway may be of various shape or combination of shapes, including without limitation, circular, elliptical, square, and triangular). The passageway may also be of variable size to accommodate the patient's needs.

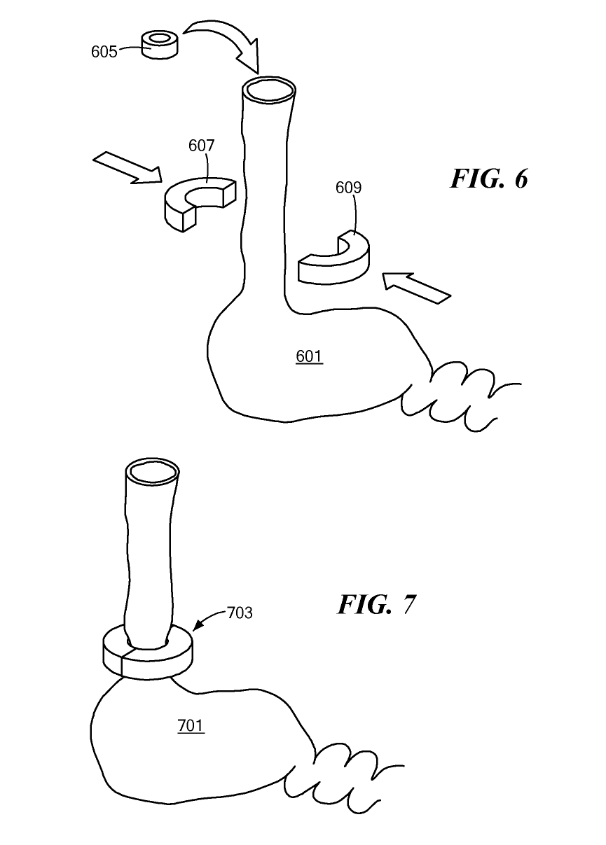

FIG. 6 shows a ring magnet and insert of an obesity management system being installed in a proper position relative to the stomach, in accordance with an embodiment of the invention. Illustratively, two ring magnet pieces 607 and 609 may be brought together around the stomach 601 during a surgical installation. The magnetic attraction between the ring magnet piece's end sections hold the ring magnet pieces 607 and 609 together to form an enclosed ring magnet 703 around the stomach 701, as shown in FIG. 7. Advantageously, no suturing or latching of the ring magnet pieces is needed. The ring magnet 703 may be manufactured to accommodate various size anatomies.

Referring to both FIGS. 6 and 7, an insert 605 may then be positioned post operationally, such that the insert 605 is magnetically held in place within the stomach 601 by the ring magnet 703. For example, in various embodiments the insert 605 may be swallowed whereupon it travels down the esophageal tract, or inserted by a catheter. Upon reaching the opening of the ring magnet 703 formed by ring magnet pieces 607 and 609 positioned around the stomach 601, the insert 605 is magnetically held in place within the stomach 601 by the ring magnet 703.

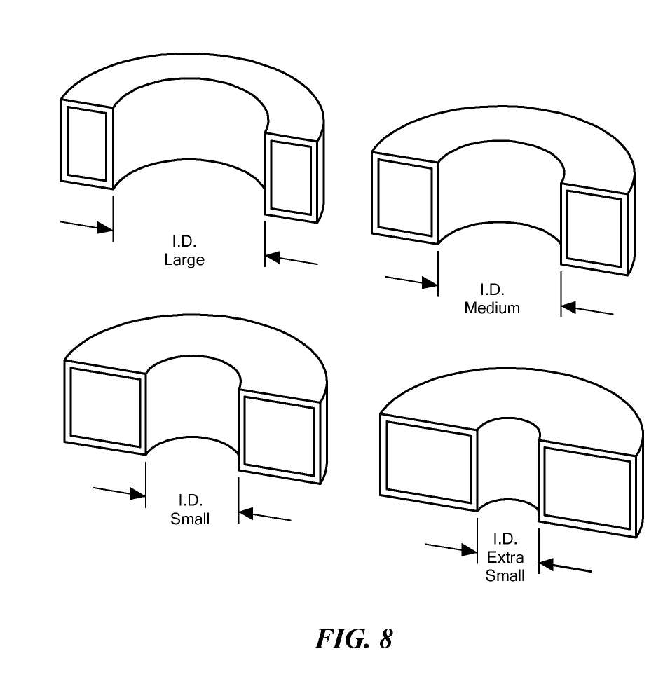

The size of the passageway formed by the insert may be varied for post operational management. FIG. 8 shows varying passageway diameters of exchangeable inserts associated with an obesity management system, in accordance with an embodiment of the invention. The passageway diameters of the inserts shown in FIG. 8 vary from large (left most insert in FIG. 8) to small (right most insert in FIG. 8). The inserts, which may be managed/installed post operationally, may be varied over time in a patient. For example, a patient may start with an insert having a small passageway diameter, resulting in a larger weight loss overtime (compared to a larger inner diameter insert). Upon, without limitation, a better lifestyle of the patient, such as a more active lifestyle, the previously inserted may be removed, and an insert with a larger passageway diameter may be installed, allowing for consumption of more food.

Upon a patient wearing an implanted magnet having to undergo Magnetic Resonance Imaging (MRI) examination, interactions between the implanted magnet and the applied external MRI magnetic field may, at high field strengths, produce potentially harmful effects. For example, the implanted magnet may experience a torque (T=m×B) that may twist the magnet out of its position, thereby injuring the implant wearer and/or destroying the mechanical fixation. Because of various risks it may be generally forbidden to undergo (at least high-field) MRI examination for patients with an implanted magnet. This may exclude the patient from certain important diagnosis methods.

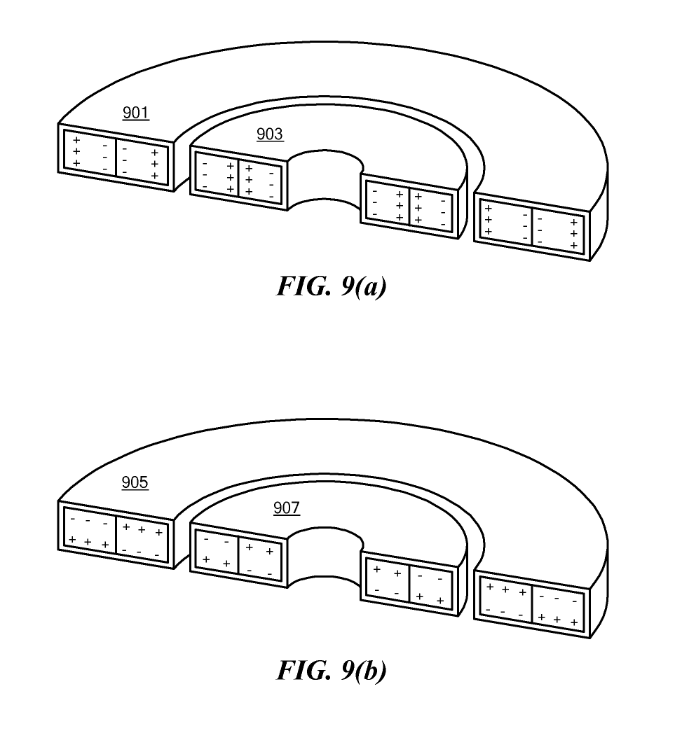

FIGS. 9(a) and 9(b) show ring magnet and insert configurations that may be advantageously used in situations where an MRI is required, in accordance with an embodiment of the invention. In particular, FIG. 9(a) show various magnets within the ring magnet 901 and/or inserts 903 mounted back to back (for, example, with either their north poles or south poles adjacent) in an anti-parallel configuration. Since the magnets have opposite magnetic moments, the total torque exerted to the arrangement in the presence of an external magnetic field of any orientation (e.g. in an MRI unit) is lessened, and in some embodiments, substantially eliminated. FIG. 9(b) shows another configuration with ring magnet 905 and/or inserts 907 that may be advantageously used.

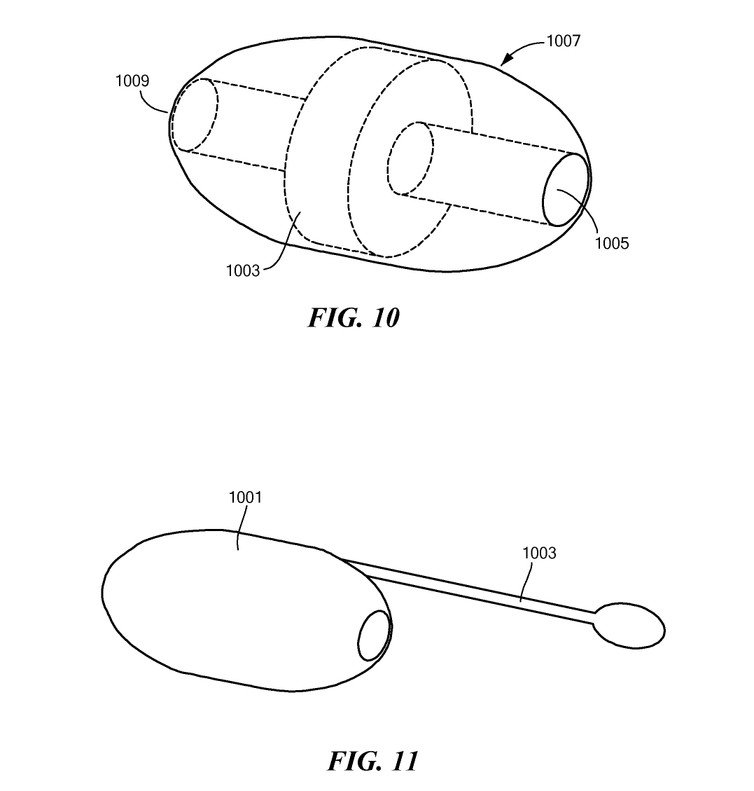

FIG. 10 shows an insert 1007 shaped to aid installation, in accordance with an embodiment of the invention. In particular, the insert 1007 is, without limitation, pill shaped, so that the insert 1007 is easily swallowed or inserted by a catheter. Other insert shapes, depending on, for example, patient anatomy, may also be implemented, such as, without limitation, spherical or cylindrical shapes. As in above embodiments, the insert 1007 includes a magnet 1003 or magnetic material such that it can be held in position by a corresponding ring magnet. The insert 1007 also includes a passageway 1009 having an inlet 1005 and outlet 1009 through which food or other substances may pass. The insert may incorporate aids for installation or extraction. For example, FIG. 11 shows an insert 1001 with an extraction tether 1003, in accordance with an embodiment of the invention.

The surface of the insert may be made of various materials that aid in installation or extraction. FIG. 12 shows an insert 1201 that includes, without limitation, a rubber encapsulation 1203, in accordance with an embodiment of the invention. The rubber encapsulation 1203 advantageously allows for easier swallowing or placement by a catheter. Other materials know in the art may also be utilized to aid in installation or extraction, such as, for example, a lubricious coating of, without limitation, polyethylene glycol (PEG).

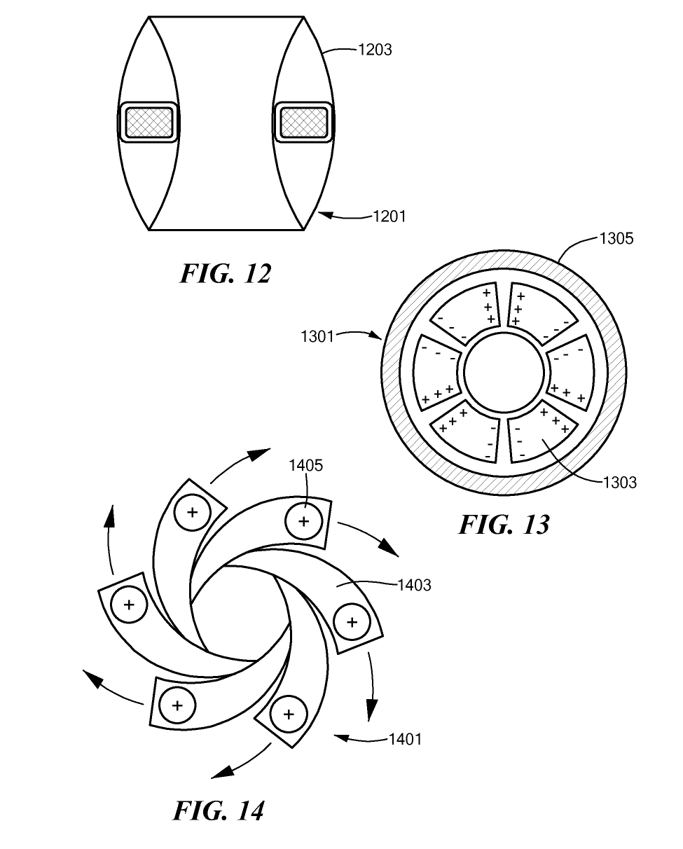

To ease in installation, the insert may include a dissolvable material that, upon dissolving, allows the insert to expand in diameter. FIG. 13 shows an insert 1301 with a dissolvable “compression” overmold 1305, in accordance with an embodiment of the invention. A ring of segmented magnets 1303 is held in a compressed position by the overmold 1305. Upon the overmold 1305 dissolving in, for example, the stomach, the ring of magnets 1303 is allowed to expand into proper position, which may be, without limitation, defined by the insert's 1301 corresponding ring magnet (not shown).

Various orientations of the magnets 1303 in the compressed position may be implemented. FIG. 14 shows an insert 1401 that includes iris shaped elements 1403 that include magnets 1405, in accordance with an embodiment of the invention. The shaped elements 1403 may initially be held in a compressed state having a reduced outer diameter by a dissolvable material overmold (not shown). Upon the material dissolving, the shaped elements 1403 are allowed to expand, driven by magnetic attraction to the insert's corresponding ring magnet.

Embodiments of the inventions are not limited to the stomach/obesity management. For example, the present invention may be employed, without limitation, into the urinary tract for incontinence management, or the larynx for various disorders of the larynx, such as pacing of the larynx.

Although various exemplary embodiment of the invention have been disclosed, it should be apparent to those skilled in the art that various changes and modifications can be made which will achieve some of the advantages of the invention without departing from the true scope of the invention. These and other obvious modifications are intended to be covered by the claims that follow.

What is claimed is:

1. A system for restricting an anatomical passageway, the system comprising:
a plurality of ring magnet pieces, each of the plurality of ring magnet pieces including:
an inner and outer surface; and
a pair of magnetic end sections between the outer surface and the inner surface, each end section having a characteristic magnetic field arrangement; and
an insert that includes at least one of a magnet and a magnetic material, wherein the ring magnet pieces cooperate due to their magnetic field arrangements to form a ring for placing around an anatomical passageway, the insert magnetically held in place by the ring magnet pieces when positioned in the anatomical passageway.

2. The system according to claim 1, wherein the anatomical passageway is associated with one of a stomach, an esophageal tract, a urinary tract and a larynx.

3. The system according to claim 1, wherein each magnetic field arrangement includes a plurality of magnetic field directions.

4. The system according to claim 1, wherein at least one of the ring magnet pieces include magnets arranged in an anti-parallel configuration.

5. The system according to claim 1, wherein the insert includes an insert passageway having an inlet and an outlet.

6. The system according to claim 1, further including a plurality of the inserts, each insert having a passageway diameter that varies from the other inserts.

7. The system according to claim 1, wherein the insert includes a tether to assist in one of extraction and installation.

8. The system according to claim 1, wherein the insert includes a plurality of magnets.

9. The system according to claim 8, wherein the magnets of the insert are arranged in an anti-parallel configuration.

10. The system according to claim 8, wherein the plurality of magnets in the insert are held by a material that dissolves within the anatomical passageway, whereupon the magnets of the insert are capable of changing position so as to increase the outer diameter of the insert.

11. The system according to claim 8, wherein the plurality of magnets in the insert are held by a material that dissolves within the anatomical passageway, and wherein the outer diameter of the insert increases upon the material dissolving and being magnetically attracted to the ring magnet pieces.

12. The system according to claim 8, wherein the plurality of magnets are arranged in an iris pattern capable of moving from a first position in which the insert has a reduced outer diameter to a second position in which the insert has an increased outer diameter.

13. The system according to claim 1, wherein the insert includes a material that dissolves within the anatomical passageway, whereupon the outer diameter of the insert increases.

14. The system according to claim 1, wherein the insert is encapsulated in a rubber.

15. A method for restricting an anatomical passageway, the method comprising:
placing a plurality of ring magnet pieces around an anatomical passageway such that due to their magnetic field arrangements they are held together to form an enclosed ring around the anatomical passageway; and
inserting a first insert in the anatomical passageway, the first insert including at least one of a magnet and a magnetic material such that the first insert is magnetically held in place by, the ring magnet pieces.

16. The method according to claim 15, wherein each ring magnet piece has an inner and outer surface, and a pair of magnetic end sections between the outer surface and the inner surface, each end piece having a characteristic magnetic field arrangement.

17. The method according to claim 16, wherein each magnetic field arrangement includes a plurality of magnetic field directions.

18. The method according to claim 15, wherein the anatomical passageway is associated with one of a stomach, an esophageal tract, a urinary tract and a larynx.

19. The method according to claim 15, wherein the anatomical passageway is associated with at least one of a stomach and an esophageal tract, the method for obesity management.

20. The method according to claim 15, wherein the insert includes an insert passageway having an inlet and an outlet.

21. The method according to claim 20, further comprising:
removing the first insert magnetically held in place by the ring magnet pieces;
inserting a second insert into the anatomical passageway such that it is magnetically held in place by the ring magnet pieces.

22. The method according to claim 21, wherein the second insert has an insert passageway that has a diameter different from the first insert.

23. The method according to claim 15, further comprising using a tether associated with the first insert to assist in one of extraction and installation.

24. The method according to claim 15, wherein the first insert includes a plurality of magnets.

25. The method according to claim 24, further comprising arranging the magnets of the first insert in an anti-parallel configuration.

26. The method according to claim 15, wherein the plurality of magnets in the first insert are held by a material that dissolves within the anatomical passageway, and wherein the outer diameter of the first insert increases upon the material dissolving.

27. The method according to claim 15, wherein the first insert includes a material that dissolves within the passageway, whereupon the outer diameter of the first insert is capable of increasing.
